(19) United States
(12) Patent Application Publication
(10) Pub. No.: US 2025/0235662 A1
(43) Pub. Date: Jul. 24, 2025

(54) MICROFABRICATED CATHETER DEVICES WITH HIGH AXIAL STRENGTH

(71) Applicant: SCIENTIA VASCULAR, INC., West Valley City, UT (US)

(72) Inventor: Clark C. Davis, Holladay, UT (US)

(21) Appl. No.: 19/175,964

(22) Filed: Apr. 10, 2025

Related U.S. Application Data
(63) Continuation of application No. 17/493,265, filed on Oct. 4, 2021, now Pat. No. 12,296,112.
(60) Provisional application No. 63/087,410, filed on Oct. 5, 2020.

(51) Int. Cl. A61M 25/00; A61M 25/01; A61M 25/09; A61M 25/10

(52) U.S. Cl. CPC A61M 25/0045; A61M 25/0053; A61M 25/001; A61M 25/0013; A61M 25/0021; A61M 25/0023; A61M 25/0026; A61M 2025/0042; A61M 25/005; A61M 25/0054; A61M 25/0108; A61M 25/0138; A61M 25/0147; A61M 25/09; A61M 2025/09108; A61M 2025/0915; A61M 25/104

(57) ABSTRACT

This disclosure describes microfabricated intravascular devices that are configured for high axial strength while also maintaining effective bending flexibility. A tube member includes a series of circumferentially extending rings connected to one another by a series of axially extending beams. Transverse cuts separate and define the rings. A series of axial cuts are aligned with the beams and extend from the beams partially into the adjoining rings so that the beam length is nested partially within the axial length of the adjoining rings. This increases the functional length of the beams to provide bending flexibility while still sufficient ring structure to provide effective axial stiffness.

### CROSS-REFERENCE TO RELATED APPLICATIONS

This application is a continuation of U.S. patent application Ser. No. 17/493,265, filed Oct. 4, 2021 and titled “Microfabricated Catheter Devices with High Axial Strength,” which claims priority to and the benefit of U.S. Provisional Patent Application No. 63/087,410, filed Oct. 5, 2020 and titled “Microfabricated Catheter Devices with High Axial Strength.” Each of the foregoing applications is incorporated herein by this reference.

### BACKGROUND

Guidewires and catheters are frequently utilized in the medical field to perform delicate procedures deep within the vasculature of the body. Typically, a catheter is inserted into a patient's femoral, radial, carotid, or jugular vessel and navigated through the patient's vasculature to the heart, brain, or other targeted anatomy. Often, a guidewire is first routed to the targeted anatomy, and one or more catheters are subsequently passed over the guidewire and routed to the desired location. Once in place, the catheter can be used to aspirate clots or other occlusions, or to deliver drugs, stents, embolic devices, radiopaque dyes, or other devices or substances for treating the patient.

In many applications, such catheters must be routed through tortuous bends and curves of a vasculature pathways to arrive at the targeted anatomy. Ideally, these catheters include design features that enable effective navigation of such tortuous pathways. For example, a catheter should be flexible enough to navigate the bends of the vasculature, but should also be able to provide sufficient pushability (i.e., the ability to transmit axial forces from proximal portions to distal portions) and torquability (i.e., the ability to transmit torque from proximal portions to distal portions).

If a catheter lacks sufficient axial stiffness, for example, it can be difficult for the operator to push the catheter forward through the vasculature. That is, axial forces applied at the proximal end by the operator may cause the catheter to axially compress and “accordion” rather than be effectively transmitted to the distal end of the catheter. Designing the catheter to have higher axial stiffness can alleviate this problem. However, increasing the axial stiffness of the catheter can cause other problems that interfere with effectiveness of the catheter. For example, increasing the axial stiffness of the catheter usually also increases the bending stiffness of the catheter, which can be detrimental if insufficient bending flexibility remains in the device.

Accordingly, there is an ongoing need for catheter devices with features that are designed to allow for effective axial stiffness without overly disrupting needed characteristics, such as the flexibility and torquability of the device.

### SUMMARY

The present disclosure describes microfabricated intravascular devices that are configured for high axial strength while also maintaining effective bending flexibility.

In one embodiment, a tube member includes a series of circumferentially extending rings connected to one another by a series of axially extending beams. A plurality of transverse cuts separate and define the rings. The transverse cuts are disposed between adjacent rings and extend in a direction transverse to the longitudinal axis of the tube member, but not so far as to completely cut through the tube member, thereby leaving beams positioned between the rings.

In some embodiments, at least a portion of the transverse cuts are wedge-shaped. For example, one or more transverse cuts may be narrower near the corresponding beams and then widen while circumferentially extending away from the corresponding beams.

In some embodiments, a series of axial cuts are aligned with the beams and extend from the beams partially into the adjoining rings so that the beam length is nested partially within the axial length of the adjoining rings. This increases the functional length of the beams to provide bending flexibility while still sufficient ring structure to provide effective axial stiffness.

In some embodiments, at least a portion of the axial cuts are wedge-shaped. For example, one or more axial cuts may be wider at an edge of the adjoining ring and then narrow while extending along the axial direction into the adjoining ring.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an exemplary catheter device that may be modified with the cut pattern described herein to provide a catheter having high axial strength;

FIG. 2A is a detailed view of a microfabricated distal section of a catheter having a conventional two-beam cut pattern;

FIGS. 2B and 2C are detailed views of microfabricated distal sections of catheters having three-beam and one-beam cut patterns, respectively;

FIGS. 3A and 3B schematically illustrate how microfabricated catheter sections can compress and “accordion” under an axial load;

FIG. 4 illustrates a microfabricated catheter section having relatively thick ring elements to enhance axial stiffness, but which concentrate bending forces in the axial beam elements; and

FIGS. 5A and 5B illustrate an exemplary microfabricated catheter section with a cut pattern that provides effective axial stiffness without overly increasing bending stiffness and thereby provides a high axial stiffness to bending stiffness ratio.

### DETAILED DESCRIPTION

### Introduction

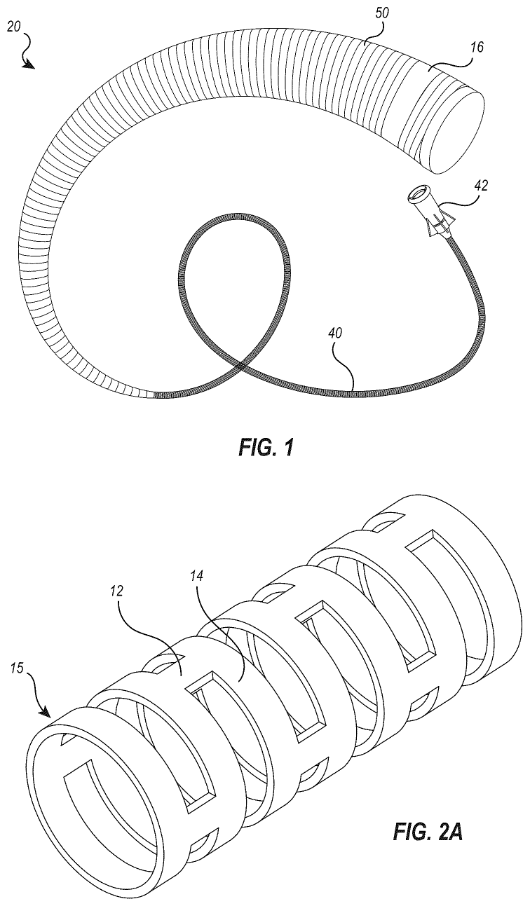

FIG. 1 illustrates an example of a conventional catheter device 10 that can be improved by incorporation of the unique, high push strength cut patterns described below. The catheter device 10 includes a proximal section 40 and a distal section 50. A radiopaque marker 16 may be located near the distal end. A hub and/or port 42 may be located at the proximal end. Due to the particular benefits of the high push strength designs in catheter applications, most of the examples described herein will refer to catheter devices. However, it will be understood that, in some embodiments, the same features may be applied to other microfabricated components of other intravascular devices such as guidewires.

At least part of the distal section 50 is microfabricated with one or more cut patterns intended to increase the effectiveness of the device. Conventionally, such cut patterns have focused on increasing the bending flexibility of the device while maintaining good torquability. However, as described below, improved cut patterns have now been designed that increase bending flexibility of the device while optimizing pushability (i.e., optimizing axial stiffness).

Although the improved cut patterns sacrifice some of the torquability of conventional cut patterns, the enhanced pushability of the device and the improved axial stiffness to bending stiffness ratios provide more effective overall functionality, particularly in applications where axial stiffness is likely to be more important that torquability, such as in many catheter applications. For example, unlike guidewires, catheters lack a solid core and therefore inherently lack good axial stiffness. Since catheters are often routed over guidewires, the guidewires may be utilized for sub-selecting vessels and reaching the anatomical target. Thus, pushability is often more important than torquability in catheters.

The length of the catheter 10 may vary according to the needs of a particular application, but will typically be within a range of about 125 cm to 175 cm. The microfabricated portion will likely vary according to particular application needs, but will typically have a length of about 50 to 90 cm. The distal-most sections (e.g., the distal-most section is about 10 to 30 cm) typically have a higher degree of microfabrication so as to be more flexible. As with catheter length, catheter diameter may vary according to application needs. Examples may range from about 2 F to 10 F, though sizes outside this range may also be utilized where suitable (e.g., outside of typical neuro and coronary applications). Aspiration catheters for use in neurovascular procedures is one exemplary application for the high push strength devices described herein.

The microfabricated section of the catheter 10 includes a plurality of cuts that extend transverse to the longitudinal axis of the catheter to form “rings” and “beams.” The rings are the circumferentially extending, ring-shaped structures and the beams are the uncut, axially extending sections of the tube that connect adjacent rings. Sections of the catheter 10 may be defined herein according to the number of beams disposed between each successive pair of rings.

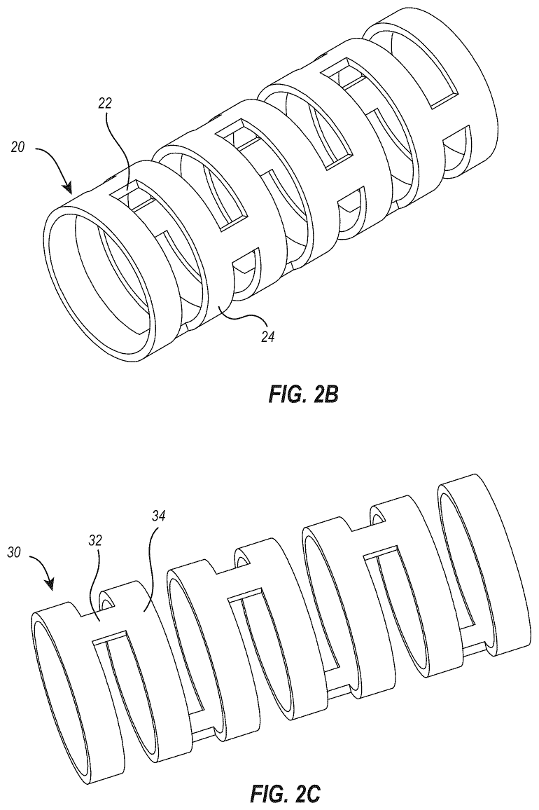

FIGS. 2A-2C illustrate conventional microfabricated configurations in order to describe general features and to define general terms. The improved features, described further below, may be applied to any of the conventional configurations shown in FIGS. 2A-2C.

FIG. 2A, for example, represents a conventional “two-beam section” 15 of a microfabricated tube member. The two-beam section includes a series of successive rings 14 and a series of beams 12 extending between and connecting the rings. As shown, each pair of adjacent rings 14 is connected by two beams 12. FIG. 2B illustrates a “three-beam section” 20 with where three of the beams 22 are disposed between each set of adjacent rings 24. FIG. 2C illustrates a “one-beam section” 30 where a single beam 32 extends between and connects each pair of adjacent rings 34. Although most of the examples described herein will reference the two-beam configuration, it will be understood that the same features may be applied to other embodiments having one-beam or three-beam configurations, or even to configurations having other numbers of beams between each set of adjacent rings.

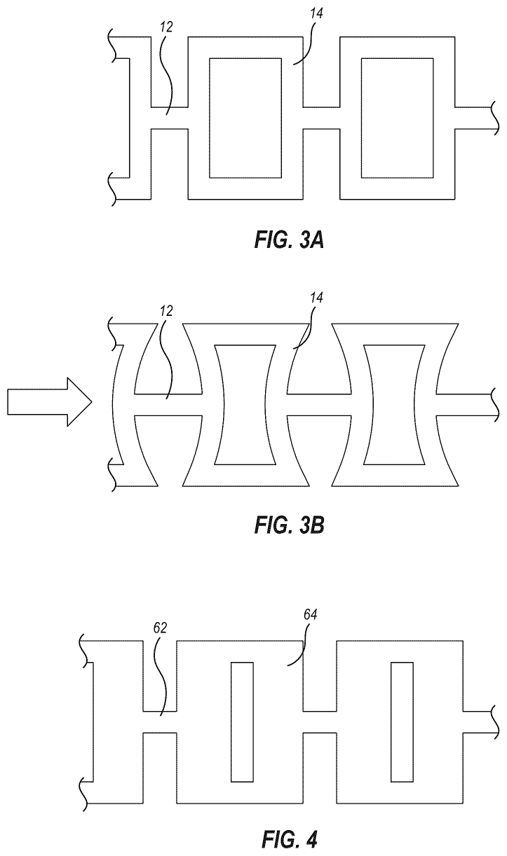

FIGS. 3A and 3B schematically illustrate how microfabricated catheter sections can compress and “accordion” under an axial load. FIG. 3A shows a side view of a conventional two-beam section (as in FIG. 2A) with beams 12 and rings 14. FIG. 3B shows that when an axial load (i.e., a push) is applied, the rings 14 may flex somewhat and absorb part of the axial load rather than fully transferring it to more distal sections of the device. This reduces the pushability of the device and makes it more difficult for the operator to track the catheter over a guidewire and/or get the catheter to the desired anatomical target.

One way to increase the axial stiffness of the device is to simply increase the length of the rings along the axial direction (sometimes this dimension is also referred to as the “thickness,” “axial length,” or “width” of the rings). FIG. 4 illustrates an embodiment with increased axial length of rings 64. While increasing the axial length of rings 64 can indeed increase the axial stiffness of the device, there is a practical limit to how much the axial length of the rings 64 can be increased. For example, if the axial length of the rings 64 is increased too much relative to the size of the beams 62, excessive bending stresses will be concentrated at the beams 62. At some point, the device will be unable to bend sufficiently without plastically deforming at the beams 62. Simply increasing the ring size until a desired axial stiffness is achieved is therefore not a practical option.

### High Push Strength Microfabricated Sections

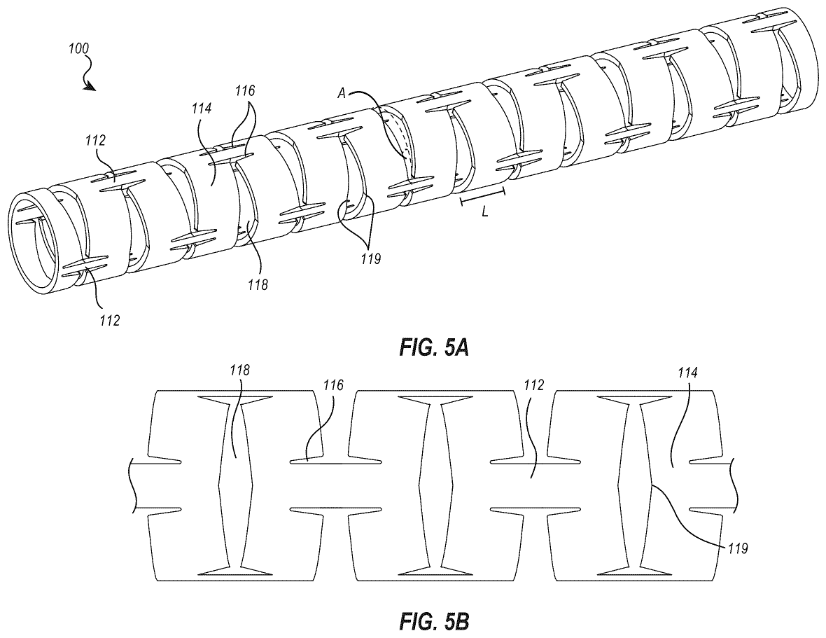

FIGS. 5A and 5B illustrate an embodiment of a high push strength cut pattern that may be applied to a tube member and utilized in a catheter device such as the device shown in FIG. 1. The cut pattern beneficially provides effective axial stiffness while still maintaining good bending flexibility. Further, unlike simply expanding the thickness of the rings, the illustrated configuration allows for high relative axial stiffness without overly concentrating stresses in the beams. FIG. 5A shows an exemplary tube member 100 (e.g., a distal section of a catheter) and FIG. 5B shows the same cut pattern if the tube member 100 were cut in half along the longitudinal axis and unrolled so as to lay flat.

As shown, the elongate tube member 100 includes a series of circumferentially extending rings 114 connected together by a series of axially extending beams 112. The rings 114 have a length “L” in the axial direction. This dimension may occasionally be referred to as the ring “width,” “axial length,” or ring “thickness,” but is typically referred to herein as the length (or more specifically the axial length) for the sake of consistency since it is the dimension parallel to the longitudinal axis of the tube member 100. Regarding the beams 112, the “length” of the beams 112 will be used herein to refer to the dimension along the axial direction, while the “width” or “thickness” of the beams will be used herein to refer to the dimension along the circumference of the tube member 100.

The rings 114 are spaced apart by transverse cuts 118 that each extend in a direction transverse to the longitudinal axis of the tube member 100 but that do not extend completely through the tube member 100. The tube member 100 is therefore somewhat similar to the conventional configurations illustrated in FIGS. 2A-2C. The illustrated tube member 100 represents a two-beam section because it includes two beams 112 between each pair of adjacent rings 114. As explained above, however, other embodiments may include configurations with a different number of beams between each pair of adjacent rings (e.g., one-beam or three-beam configurations).

Unlike the conventional configurations shown in FIGS. 2A-2C, however, the illustrated embodiment also includes a series of axial cuts 116 aligned with the beams 112. Each axial cut 116 starts along an edge of a corresponding beam 112 and extends partially into the adjoining ring 114 in a substantially axial direction so that the corresponding beam 112 is partially “nested” within the axial length of the adjoining beam 112.

The beams 112 of the illustrated embodiment in FIGS. 5A and 5B are each associated with axial cuts 116 that extend into each of the adjoining rings 114, which represents a preferred embodiment. However, other embodiments may include fewer axial cuts 116. For example, some embodiments may have beams that are only associated with axial cuts extending into one of the two adjoining rings (e.g., just the proximal adjoining ring, or just the distal adjoining ring, but not both). In another embodiment, the tube member 100 may have some beams 112 that are associated with axial cuts 116 while other of beams 112 are not associated with axial cuts 116.

Because part of the beam 112 is nested within the axial length of the ring 114, the result is a more flexible beam structure per unit length of the tube member 100 as compared to the same structure without the axial cuts 116. In other words, the illustrated cut pattern provides additional functional length to the beams 112, and thus greater bending flexibility to the device, while still allowing the rings 114 to be relatively thick (i.e., to have relatively long axial length) along most of the circumference of the device. The overall structure is therefore capable of providing good axial stiffness without overly increasing the bending stiffness, resulting in devices that have a favorable axial stiffness to bending stiffness ratio.

As shown in FIGS. 5A and 5B, the transverse cuts 118 may have a wedge-shape. The wedge-shaped cuts 118 beneficially provide additional clearance allowing the device to bend along an inside of a curve. Similarly, for embodiments that fill the gaps of the device with a polymer material, such as discussed further below, the wedge-shaped cuts 118 provide additional room for the polymer that gets compressed toward the inside of a curve.

For example, at a given axial position of the tube member 100, the transverse cuts 118 may be narrower near the beams 112 and then widen while extending away from the beams 112. Starting from one of the beams 112 and extending around the circumference, the cuts 118 can widen until reaching an apex 119 and can then begin to narrow again while continuing to extend toward the opposite beam 112. As shown, the apex 119 may be located at a location equidistant from the two beams 112, though in other embodiments one or more transverse cuts 118 may be asymmetrical and the apex 119 need not be equidistant from each beam 112.

The size and shape of the wedge-shaped transverse cuts 118 may be varied. In general, wider gaps provide greater clearance for tighter bending but at the cost of a reduction in axial stiffness. Accordingly, the wedge angle and/or gap size may be increased for applications requiring greater bending flexibility, or the wedge angle and/or gap size may be reduced for applications requiring greater axial stiffness. Alternatively, the wedge angle and/or gap may be increased for areas of the device requiring greater flexibility and reduced for areas of the device requiring greater axial strength. In one nonlimiting example, the wedge angle and/or gap size may be increased at more distal sections of the device relative to more proximal sections of the device. In certain embodiments, at least at a distal section of the tube member 100, the gap size of the apex 119 (i.e., the widest part of the transverse cut 118) may be about 25% to about 100% the length of the rings 114, or about 35% to about 75% the length of the rings 114. Additionally, or alternatively, the wedge angle may increase or decrease progressively between one section to another such that there is a gradual change in wedge angle from a first section to a second section.

The angle “A” at which the wedge-shape of the transverse cuts 118 extends from the beam 112 may range from about 2 degrees to about 35 degrees, or about 5 degrees to about 25 degrees, or about 10 degrees to about 20 degrees. In other words, if an angle of 0 degrees represents a straight, perpendicular cut, the wedge-shaped cuts 118 preferably have an angle greater than 0 degrees but less than about 35 degrees, more typically less than about 25 degrees or less than about 20 degrees.

In the illustrated embodiment, the transverse cuts 118 angle in both axial directions (proximally and distally). That is, starting at a given beam 112, and moving in a circumferentially perpendicular direction around the tube member 100 toward another beam 112, the corresponding transverse cut 118 is angled away from perpendicular along both the proximally adjoining ring 114 and the distally adjoining ring 114 Other embodiments may include transverse cuts 118 that are only angled away from perpendicular in one direction (i.e., along only the proximally adjoining ring or only along the distally adjoining ring).

One or more axial cuts 116 may be wedge-shaped. As shown in FIGS. 5A and 5B, the axial cuts 116 may be somewhat wider where the cuts “start” along the edge of the ring, and then narrow while extending along the axial direction farther into the adjoining ring. As with the wedge-shape of the transverse cuts 118, the wedge shape of the axial cuts 116 may provide additional clearance allowing more movement of the rings 114 relative to the beam 112 allowing the rings 114 to better bend towards one another along the inside of a curve when the tube member 100 is bent. If a straight axial cut (parallel to the longitudinal axis) has a cut angle of 0 degrees, the angle of the axial cuts 116 may be greater than 0 degrees but less than about 35 degrees, more typically less than about 25 degrees or less than about 20 degrees.

At least for distal sections of the tube member 100, the axial cuts 116 may extend into the adjoining rings 114 a distance equal to about 25% to about 75%, or about 35% to about 65%, or about 45% to about 55% of the axial length of the rings 114. The greater the axial cuts 116 extend into the rings 114, the greater the added functional length of the associated beam 112. However, this comes at the expense of some of the structure of the rings, and thus deeper axial cuts 116 reduce some of the structure of the ring 114 otherwise contributing to axial stiffness, at least at the particular portion of the ring 114 coincident with the axial cuts 116 and the beam 112. In some applications, the axial cuts 116 may extend farther into the rings 114 to increase the length of the associated beam 112 and thus the flexibility of the tube member 100. In other applications, the axial cuts 116 may extend farther into the ring 114 on one section of the device relative to another section of the device. For example, the axial cuts 116 may be increased or decreased near the distal or proximal ends of the device.

Accordingly, the beam length may also increase or decrease in size. This may be a result of the length of the axial cut 116 as discussed above. Alternatively, or in addition, the beam length may vary in size independent of the axial cut 116 by increasing or decreasing the length of the portion of the beams 112 between the corresponding pair of rings 114. In some application, one section of the tube member 100 may have a beam length which is longer relatively than the beam length of another section in order to give the device differential flexibility on different parts of the device. Finally, the beam length may vary progressively from a first section to a second section such that the beam length increases or decreases gradually between the two sections.

The beam width, or the beam thickness, may also vary in size according to the application of the device, the overall size of the device, and/or the section of the device. In some applications, one section of a device may have a beam width that is larger relatively than a second section of the device. In addition, the beam width may vary progressively such that the beam width of each beam 112 increases gradually between a first section and a second section.

The ring size may also vary according to the overall size of the device and/or the section of the device. For example, at a distal section of the tube member 100, the rings 114 may have a ring length to ring diameter ratio of about 0.25 to 0.8, or about 0.35 to 0.65, or about 0.4 to 0.6. In some applications, the entire device will have a utilize similar ring sizes with each ring 114 having similar axial length. Alternatively, in some applications, the ring size of one or more sections of the device will differ from one or more other sections of the device such that one or more sections of the device have greater axial strength relative to one or more other sections. In some embodiments, the ring axial length may vary along the device progressively, such that the ring size increases or decreases gradually from one section to another section.

As shown, the beams 112 between each pair of adjacent rings 114 may be equally circumferentially spaced (e.g., spaced by 180 degrees in a two-beam configuration), although other embodiments may arrange the beams so as not to be equally circumferentially spaced. Sets of beams 112 may also be rotationally offset from adjoining sets of beams 112. For example, a set of beams 112 between a given pair of adjacent rings may be rotationally offset from the set of beams of a previous and/or subsequent pair of adjacent rings. In the illustrated embodiment, the rotational offset is 90 degrees. That is, a first pair of beams is provided at a first rotational position, then while moving along the length of the tube member 100 the next pair of beams is offset from the first pair by 90 degrees.

Other rotational offsets may be utilized. The rotational offset may be about 5 degrees to about 90 degrees, for example. A rotational offset that is less than 90 degrees provides a helical pattern that minimizes preferred bending axes in the tube member 100. Other beneficial “distributed” beam arrangements may alternatively be utilized to avoid preferred bending axes. These are described in more detail in U.S. patent application Ser. No. 16/616,139, entitled “Micro-Fabricated Medical Device Having a Non-Helical Cut Arrangement,” which is incorporated herein by this reference in its entirety.

The tube member 100 may be formed from any material or combination of materials suitable for an intravascular application. Examples include polymer materials such as polyether ether ketone (PEEK), other polymers that can be formulated with a similar range for modulus of elasticity, stainless steel, or superelastic materials such as nitinol. Preferred embodiments are formed from nitinol.

As briefly mentioned above, a polymer material may be added to the tube member 100 to fill in the gaps made by transverse cuts 118 and axial cuts 116 and allow the tube member 100 to be transport fluids. The polymer material may comprise an elastomer such as a polyether block amide and/or another similar polymer.

Another advantage of the described embodiments as compared to the conventional configurations relates to the relatively lower open gap space along the outer surface of the tube member 100. Because the improved cut patterns allow for increased axial length of the rings, less of the overall outer surface area is taken up by gaps. This means that proportionally less of the device relies on the polymer material for maintaining fluid tight integrity under pressure and thus the device is less likely to fail when delivering fluids under pressure.

Other embodiments may omit a polymer material. For example, certain applications may not require the delivery or aspiration of fluids and can feasibly utilize a device where the gaps are not filled. Keeping the gaps open is beneficial, in certain applications, because adding polymer to the transverse and axial cuts increases the bending stiffness of the tube member 100. Other embodiments may utilize one or more liners rather than a polymer fill material. For example, an inner liner may be disposed along an inner surface of the tube member 100 and/or an outer liner may be disposed along an outer surface of the tube member 100. Either way, the inner liner and the outer liner do not fill the gaps of the tube member 100. Such embodiments may advantageously keep the gaps of the transverse and axial cuts open and unobstructed, which reduces the amount of resistance to bending and thereby allows for lower bending stiffness.

It should be understood that the foregoing features are primarily directed toward a distal section of the tube member 100. Similar features may be utilized in more proximal sections. However, more proximal sections typically do not require the same bending flexibility and so such sections may be tailored more toward pushability and/or torquability and less toward bending flexibility. Thus, more proximal sections may be modified via one or more of increasing the axial lengths of the rings, increasing the width of the beams, decreasing the size of the wedge-shaped gaps, decreasing the depth of the axial cuts, or increasing the number of beams between each pair of rings.

### Examples

A useful metric for comparing intravascular devices is the ratio of axial stiffness to bending stiffness. Axial stiffness and bending stiffness (i.e., flexural rigidity) are typically reported using different units. In the SI system, for example, axial stiffness is typically reported in units of force per distance (e.g., Newtons per meter), while bending stiffness is typically reported in units of force times distance squared (e.g., Newtons times meters squared). When using such units, a useful metric can be determined by comparing the ratio of the axial stiffness to the bending stiffness of a micromachined structure and comparing that number to the ratio of axial stiffness to bending stiffness of a homogenous material (not micromachined but otherwise similar to the micromachined structure). For example, the ratio of axial stiffness to bending stiffness of the micromachined structure can be divided by the ratio of axial stiffness to bending stiffness of the homogenous material to provide a useful comparative ratio illustrating how the micromachined structure compares to a baseline homogenous material. Such an overall ratio is unitless. This metric is referred to herein as the micromachined-to-homogenous ratio.

A variety of catheter devices and materials were tested to measure ratios of axial stiffness to bending stiffness. Materials tested included tubes of homogenous rubber and plastic materials, including PEBAX® (a polyether block amide), polyurethane, and the like. Commercial catheter devices formed with sections of coil and/or braided material were also tested. The micromachined-to-homogenous ratio for commercially available catheters typically ranged from about 1 to 2.5. The highest micromachined-to-homogenous ratios were found in certain commercial catheter products with coil and/or braided sections and were measured to be about 3.

In comparison with the above, tube members formed with a high push strength configuration as shown in FIG. 5A were also tested. Tube members formed from nitinol were the most preferred, though tube members formed of other materials also performed well. The high push strength configuration provided micromachined-to-homogenous ratios that were significantly higher than those for common coil and/or braid arrangements. Tube members with the high push strength configuration had micromachined-to-homogenous ratios greater than 3, and in some instances much greater than 3. Certain tests showed micromachined-to-homogenous ratios of about 14. Certain tests even showed micromachined-to-homogenous ratios of up to 100 at distal sections of the tube having high degrees of microfabrication.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An elongate tube member for an intravascular device, the elongate tube member extending along a longitudinal axis and comprising:
a plurality of circumferentially extending rings, each ring having an axial length;
a plurality of transverse cuts each positioned between adjacent rings, each transverse cut extending in a direction transverse to the longitudinal axis of the tube member;
a plurality of axially extending beams each extending from one ring to another to connect adjacent rings; and
a plurality of axial cuts, the plurality of axial cuts including
(i) proximal cuts that, for each beam, begin at an edge of an adjoining proximal ring and extend partially into the adjoining proximal ring in a substantially proximal axial direction so that the beam is at least partially nested within the length of the adjoining proximal ring, and
(ii) distal cuts that, for each beam, begin at an edge of an adjoining distal ring and extend partially into the adjoining distal ring in a substantially distal axial direction so that the beam is at least partially nested within the length of the adjoining distal ring,
wherein the proximal cuts and/or distal cuts of the plurality of wedge-shaped axial cuts extend farther into the ring on a first section of the elongate tube member relative to a second section of the elongate tube member,
wherein the first section of the elongate tube member is positioned proximally or distally of the second section, and
wherein the first section of the elongate tube member has a flexibility different than a flexibility of the second section of the elongate tube member.

2. The tube member of claim 1, wherein at least a portion of the transverse cuts are wedge-shaped.

3. The tube member of claim 2, wherein at least a portion of the transverse cuts are narrower near the corresponding beams and widen while circumferentially extending away from the corresponding beams.

4. The tube member of claim 1, wherein at least a portion of the axial cuts are wedge-shaped.

5. The tube member of claim 4, wherein at least a portion of the axial cuts are wider at an edge of the adjoining ring and narrow while extending along the axial direction into the adjoining ring.

6. The tube member of claim 1, wherein the tube member has a two-beam configuration such that there is a pair of beams between each pair of adjacent rings, and the pair of beams between each pair of adjacent rings are circumferentially spaced by about 180 degrees.

7. The tube member of claim 6, wherein the two-beam configuration includes a rotational offset such that the beams between a given pair of adjacent rings are rotationally offset from beams of a previous and/or subsequent pair of adjacent rings.

8. The tube member of claim 7, wherein the rotational offset is about 5 degrees to about 90 degrees.

9. The tube member of claim 1, wherein axial lengths of the rings progressively decrease toward a distal end of the tube member.

10. The tube member of claim 1, wherein thicknesses of the beams progressively decrease toward a distal end of the tube member.

11. The tube member of claim 1, wherein at a distal section of the tube member, the rings have a ring length to ring diameter ratio of about 0.25 to 0.8.

12. The tube member of claim 1, wherein at least a section of the tube member has a micromachined-to-homogenous ratio of at least 3.

13. The tube member of claim 1, wherein the tube member is formed from one or more of polyether ether ketone (PEEK), stainless steel, or nitinol.

14. The tube member of claim 1, further comprising a polymer applied to the tube member to fill in the transverse cuts and the axial cuts.

15. The tube member of claim 1, further comprising one or both of an inner liner or an outer liner.

16. The tube member of claim 15, wherein one or both of the inner liner or outer liner do not fill in the transverse cuts or the axial cuts.

17. The tube member of claim 1, wherein the beam length increases or decreases from a first section of the tube member to a second section of the tube member.

18. An elongate tube member for an intravascular device, the elongate tube member extending along a longitudinal axis and comprising:
a plurality of circumferentially extending rings, each ring having an axial length;
a plurality of transverse cuts each positioned between adjacent rings, each transverse cut extending in a direction transverse to the longitudinal axis of the tube member;
a plurality of axially extending beams each extending from one ring to another to connect adjacent rings; and
a plurality of axial cuts, the plurality of axial cuts including
(i) proximal cuts that, for each beam, begin at an edge of an adjoining proximal ring and extend partially into the adjoining proximal ring in a substantially proximal axial direction so that the beam is at least partially nested within the length of the adjoining proximal ring, and
(ii) distal cuts that, for each beam, begin at an edge of an adjoining distal ring and extend partially into the adjoining distal ring in a substantially distal axial direction so that the beam is at least partially nested within the length of the adjoining distal ring,
wherein the proximal cuts and/or distal cuts of the plurality of wedge-shaped axial cuts extend farther into the ring on a first section of the elongate tube member relative to a second section of the elongate tube member,
wherein the first section of the elongate tube member is positioned proximally or distally of the second section,
wherein the first section of the elongate tube member has a flexibility different than a flexibility of the second section of the elongate tube member, and
wherein axial lengths of the rings progressively decrease toward a distal end of the tube member and/or thicknesses of the beams progressively decrease toward the distal end of the tube member.

19. The tube member of claim 18, wherein at least a portion of the transverse cuts are wedge-shaped.

20. The tube member of claim 18, wherein at least a portion of the axial cuts are wedge-shaped.
